(19) United States
(12) Patent Application Publication
(10) Pub. No.: US 2025/0145538 A1
(43) Pub. Date: May 8, 2025

(54) Method for preparing Fly Ash for Incorporation into Concrete

(71) Applicant: Christopher Calva, SR., Fort Worth, TX (US)

(72) Inventor: Christopher Calva, SR., Fort Worth, TX (US)

(21) Appl. No.: 18/219,601

(22) Filed: Jul. 7, 2023

Related U.S. Application Data
(60) Provisional application No. 63/339,808, filed on May 9, 2022.

(51) Int. Cl. C04B 40/00; C04B 14/06; C04B 14/10; C04B 18/08; C04B 28/04; G01N 33/38

(52) U.S. Cl. CPC C04B 40/0032; C04B 14/06; C04B 14/106; C04B 18/08; C04B 28/04; G01N 33/383

(57) ABSTRACT

A system and method for amending out of specification ash to a suitable amended addition material to a concrete mixture includes analyzing a unit quantity of out of specification ash to determine concentrations of each of calcium oxide, sulfur trioxide, and unburnt carbon. The out of specification ash is sieved to determine fineness. Based upon measured concentrations and determined fineness, a mixture is designed to add materials in design-determined quantities of each of quicklime, kaolin, masonry silica blend, manufactured sand and in specification fly ash. By adding each of the quicklime, kaolin, masonry silica blend, manufactured sand and in specification fly ash in each of the design-determined quantities, creating an amalgamated blend of added materials to form an amended addition material. The amended addition material is substituted for a selected quantity of Portland cement in the concrete mixture.

### FIELD OF INVENTION

This disclosure relates to the field of compounding concrete with pozzolans and, more specifically, to exploit substandard fly ash as a pozzolan.

### BACKGROUND

Concrete is made of cement, aggregates (inert granular materials such as sand, gravel, or crushed stone), and water. Most modern concrete is a mix of Portland cement—limestone, sandstone, ash, chalk, iron, and clay, among other ingredients, heated to form a glassy material that is finely ground—with so-called “aggregates.” These aggregates, usually sand or crushed stone, are not intended to chemically react because if they do, they can cause unwanted expansions in the concrete. The nucleation of concrete is a very highly balanced reaction wherein the addition of chemicals can each affect the resulting concrete when fully cured.

The first calcium silicate cements were produced by the Greeks and Romans, who discovered that volcanic ash, if finely ground and mixed with lime and water, produced a hardened mortar, which was resistant to weathering. The reaction is known as the pozzolanic reaction and it is the basis of the contribution made to strength and concrete performance by materials such as fly ash, microsilica and metakaolin in modern concrete.

In the mid-eighteenth-century John Smeaton discovered that certain impure limes (these contained appropriate levels of silica and alumina) had hydraulic properties. That is, they contained reactive silicates and aluminates, which could react with water to yield durable hydrates, which resisted the action of water. Smeaton used this material in the mortar used to construct the Eddystone Lighthouse in 1759.

The term ‘Portland cement’ was first applied by Joseph Aspdin in his British Patent No. 5022 (1824), which describes a process for making artificial stone by mixing lime with clay in the form of a slurry and calcining (heating to drive off carbon dioxide and water) the dried lumps of material in a shaft kiln. The calcined material (clinker) was ground to produce cement. The term ‘Portland’ was used because of the similarity of the hardened product to that of Portland stone from Dorset and because this stone had an excellent reputation for performance.

Joseph Aspdin was not the first to produce a calcium silicate cement but his patent gave him the priority for the use of the term ‘Portland cement’. Other workers were active at the same time or earlier, most notably Louis Vicat in France. Blezard (1998) gives a comprehensive review of the history of the development of calcareous (lime-based) cements. The cements produced in the first half of the nineteenth century did not have the same compound composition as modern Portland cements as the temperature achieved was not high enough for the main constituent mineral of modern cements, tricalcium silicate (C₃S), to be formed. The only silicate present was the less reactive dicalcium silicate (C₂S).

In the last 150 years, experience with the addition of various supplementary cementing materials (e.g., fly ash, silica fume) and chemical admixtures has guided the selection of these supplements which must be selected judiciously to achieve specified structural properties. These supplementary ingredients incorporated in concrete function in different ways, acting as either a filler or a binder. The binder (cement paste) “glues” the filler together to form a synthetic conglomerate. The constituents used for the binder are cement and water, while the filler can be fine or coarse aggregate. Aggregates are generally chemically inert, and act as solid bodies held together by the cement. Aggregates come in various shapes, sizes, and materials ranging from fine particles of sand to large, coarse rocks.

Because ordinary Portland cement (“OPC”) is the most expensive ingredient in making concrete, economies drive suppliers to minimize the amount of OPC used. One approach is to substitute as much as 70 to 80% of the volume of OPC used in the concrete mix as aggregate; the selected inert aggregate displacing a similar volume of OPC, and thereby keeping the cost of the resulting concrete mix low. The selection of an aggregate is determined, in part, by the desired characteristics of the concrete. For example, the density of concrete is determined by the density of the aggregate. Soft, porous aggregates can result in weak concrete with low wear resistance, while using hard aggregates can make strong concrete with a high resistance to abrasion.

Water, also, is a key ingredient, which when mixed with OPC, forms a paste that binds the aggregate together. The water causes the hardening of concrete through a process called hydration. Hydration is a chemical reaction in which the major compounds in OPC and such other cementitious materials in the mix form chemical bonds with water molecules commonly referred to as hydrates or hydration products. Because the resulting concrete must be both strong and workable, a careful balance of the OPC and cementitious materials to water ratio is required when making viable concrete.

Cementitious materials are divided into two categories based on the type of reaction they undergo: either hydraulic or pozzolanic. Hydraulic materials are characterized by the most common, OPC which reacts directly with water to form cementitious compounds. In contrast, pozzolanic materials chemically react with calcium hydroxide, a soluble hydration product, in the presence of moisture to form compounds possessing cementitious properties. A pozzolana is a natural or artificial material containing silica in a reactive form. By themselves, pozzolanas have little or no cementitious value. However, in a finely divided form and in the presence of moisture they will chemically react with alkalis to form cementing compounds. Pozzolanas must be finely divided to expose a large surface area to the alkali solutions for the reaction to proceed. Examples of pozzolanic materials are volcanic ash, pumice, opaline shales, burnt clay and fly ash. The silica in a pozzolana must be amorphous, or glassy, to be reactive. Pozzolanas increase the resistance of concrete against environmental attack since they reduce permeability, absorption and ion diffusivity in the cured concrete product. Pozzolanic reactions occur over long-time scales (months to years).

Concrete is generated via a chemical hydration reaction between cement and water. OPC grains are dissolved in a mixture of water sand, and small rock particles, releasing calcium and silicon ions in the process. Once the ions spread throughout the mixture and reach a critical concentration in solution, they begin to precipitate out of solution and form a cement film around the small rock particles. This cycle of dissolution, diffusion, and precipitation continues for about an hour, causing the film to grow and the concrete to strengthen until the precipitated grains begin to impinge on one another and the entire mixture solidifies. It is at this point that the concrete begins to “set” and lose its fluid properties. Hydration of concrete is visually complete in only a few hours; however, on the microscopic level, hydration can continue to occur for months or even years. Such is the hydraulic reaction.

The hydration of OPC involves the reaction of the anhydrous calcium silicate and aluminate phases with water to form hydrated phases. These solid hydrates occupy more space than the anhydrous particles and the result is a rigid interlocking mass whose porosity is a function of the ratio of water to cement (w/c) in the original mix. Provided the mix has sufficient plasticity to be fully compacted, the lower the w/c, the higher will be the compressive strength of the hydrated cement paste/mortar/concrete and the higher the resistance to penetration by potentially deleterious substances from the environment. Cement hydration is complex, and it is appropriate to consider the reactions of the silicate phases (C₃S and C₂S) and the aluminate phases (C₃A and those of the various ferrites, C₄AF collectively) separately. Setting is largely due to the hydration of C₃S and it represents the development of hydrate structure, which eventually results in compressive strength.

The C—S—H gel which forms around the larger C₃S and C₂S grains is formed in situ and has a rather dense and featureless appearance when viewed using an electron microscope. This C—S—H gel is formed initially as reaction rims on the unhydrated material but as hydration progresses the anhydrous material is progressively replaced and only the largest particles (larger than ˜30 microns) will retain an unreacted core after several years of hydration. This dense hydrate is referred to as the ‘inner product’.

The ‘outer hydration product’ is formed in what was originally water-filled space and also space occupied by the smaller cement grains and by interstitial material (C₃A and C₄AF). When viewed using an electron microscope this material can be seen to contain crystals of Ca(OH)₂. The structure of the outer product is strongly influenced by the initial water-to-cement ratio, which in turn determines paste porosity and consequently strength development.

Because of the more immediate hydraulic reaction, it is the OPC that hardens initially and the pozzolans, if present, fill voids and strengthen the resulting matrix. The pozzolanic activity is a measure for the degree of reaction over time or the reaction rate between a pozzolan and Ca₂⁺ or calcium hydroxide (Ca(OH)₂) in the presence of water. The rate of the pozzolanic reaction is dependent on the intrinsic characteristics of the pozzolan such as the specific surface area, the chemical composition and the active phase content. It is the main reaction involved in the Roman concrete invented in Ancient Rome and used to build, for example, the Pantheon. The pozzolanic reaction converts a silica-rich precursor with no cementing properties, to a calcium silicate, with good cementing properties. The high surface area aluminate and silicate minerals are pozzolan phases, which in the presence of water and an alkali (e.g., calcium) produce cementitious materials, comprising calcium silicates and aluminate hydrates.

Fly ash is acceptable for use in limited quantities as a pozzolanic additive to OPC in concrete applications such as buildings and highways in the United States for most operating agencies if the fly ash should meet specifically the standard laid out in ASTM C618; this is the “spec” which characterizes “in spec” fly ash. While ASTM C618 is a useful specification, it has prevented the exploration of distinct formulations of concrete with what, by the definition set forth therein must be rejected for any supervised contract. FIG. 1A is a table that summarizes the regulatory parameters according to ASTM C618. Importantly, this regulation has been incorporated as the standard for structural concrete by nearly all contracting agencies for any proposed projects.

Pozzolanic materials are commonly classified according to their origins as artificial or natural materials. One such material is fly ash derived from burning coal, especially in modern incinerators. The American Society for Testing and Material (ASTM) International classifies artificial fly ash pozzolanic material as either of Class F and Class C materials. Class F fly ash is pozzolanic, lacking significant CaO and, thus, having little or no cementing value alone. Class C fly ash has more CaO giving it self-cementing properties as well as pozzolanic properties. Class C ash is thus referred to as high calcium fly ash because it typically contains more than 20 percent CaO. Class F ashes are typically derived from bituminous and anthracite coals and consist primarily of an alumino-silicate glass, with quartz, mullite, and magnetite also present. Natural pozzolanic materials, or natural pozzolans (NPs), are classified as Class N materials. Class N materials include diatomaceous earth; opaline cherts and shales; calcined or uncalcined tuffs and volcanic ashes (VAs); and some clays and shales that require calcination to induce satisfactory properties. Any ash that does not fit within these three categories are not used as they are “out of spec” ash and are, generally disposed of in ponds or landfills.

Natural pozzolanic materials, such as volcanic ashes (“VA”), can be used as supplementary cementitious materials. VA was an ingredient used by the Romans in the mixing of their concrete over 1,500 years ago and many of the resulting structures are still standing today—a testament to the mixture's durability. They would make the concrete by first mixing volcanic ash with lime and seawater to make mortar, which is later incorporated into chunks of volcanic rock, the ‘aggregate’. The combination produces a so-called pozzolanic reaction, so named after the city of Pozzuoli in the Bay of Naples.

Fly ash is produced from the burning of pulverized coal in boilers. Pulverized coal firing ensures complete combustion of coal, thus ensuring higher efficiency of steam generators. It also characterizes the resulting ash. The finer the grinding of coal, the more efficient its combustion and thus the resulting ash is also finer. Fly ash typically consists of spherical glassy particles, either hollow or filled, ranging in size from 0.1 to 100 μm (3.94×10⁻³ in.) with a bulk density of 0.54 to 0.86 g/cm³ (33.71 to 53.69 lb./ft.³), specific gravity of 2.1 to 3.0, and a specific surface area between 0.3 and 1.0 m²/g. The bulk chemical composition of fly ash can vary quite significantly but is generally dominated by silicate, aluminate, iron, and calcium phases with minor quantities of alkalis, alkali earth metals, and other metals. Advantageously, these are components of Tobermorite. SiO₂ forms calcium silica hydrates (tobermorite), dicalcium silicate (C₂S), and tricalcium silicate (C₃S), which are the main strength-contributing components of the pozzolanic behavior. Setting and hardening behavior, strength and dimensional stability depend primarily on the tobermorite gel. Development of the microstructure of hydrated cement occurs after the concrete has set and continues for months (and even years) after placement.

Fly ash, however, has recently diminished in quality and changed in its makeup. In the quest to reduce their individual carbon footprints nationwide, utility coal plants, in response to the pressures and mandates of the Obama Administration to reduce airborne pollutants, the utility plants have been injecting activated carbon into flue stacks to capture mercury liberated in combustion. Because of this change in the combustion, the resulting fly ash now contains elevated level of carbon and carbon compounds. This described increase in fly ash carbons causes a loss in the compressive strength of concrete including, especially, the increased air content in formed concrete. As such, this included carbon compromises the resulting concrete mixes by moving the entrained air outside of acceptable specifications. Thus, the carbon content of the fly ash must be measured to ensure the integrity of the resulting mix, and without adding sufficient silica to offset this greater carbon content, the effects of additional carbon are almost impossible to mitigate.

The chemistry of cement is highly complex. Lime (CaO) in the presence of silica (SiO₂), alumina (Al₂O₃), and iron oxide (Fe₂O₃) forms sulfoalumina hydrates (ettringites), and calcium silica hydrate (tobermorite) combined are the pozzolanic end-products. By convention, the annotation of the chemistry is greatly simplified using a reduced nomenclature. The four ingredients that matter in any cement are expressed in this nomenclature.

| | | |
|---|---|---|
| a. Lime | CaO | = C |
| b. Alumina | Al₂O₃ | = A |
| c. Silica | SiO₂ | = S |
| d. Water | H₂O | = H |

As stated above, tobermorite is a calcium silicate hydrate mineral; it has a chemical formula: Ca₅Si₆O₁₆(OH)₂·4H₂O or Ca₅Si₆(O,OH)₁₈·5H₂O. A combination of diffusion-transport effects and chemical reactions promotes the alteration of the microstructure of the material when subject to a flow of water: dissolution of cement constituents such as portlandite (calcium hydroxide, denoted CH in cement notation) and calcium silicate hydrate (C—S—H). The C—S—H gel, which constitutes at least 60% of the fully hydrated cement paste by volume, is the main strength-giving phase, also responsible for the durability and radionuclide barrier properties of cement owing to the features of its microstructure (porous structure and alkaline solution inside the pores that limit the solubility of radionuclides).

There is often some amount of unburnt carbon in fly ash, which has a strong effect on resulting admixtures such as interfering with air-entraining admixtures (AEA). AEAs facilitate the development of a system of microscopic air bubbles within concrete during mixing. AEAs increase the freeze-thaw durability of concrete, increase resistance to scaling caused by deicing chemicals, and improve workability. Therefore, it is important to determine a percentage of this unburnt carbon in the fly ash, as it affects air entrainment and, subsequently, determines resistance to freezing and thawing.

Fly ash, especially Class F fly ash, is effective in three ways in substantially reducing alkali-silica expansion: 1) it produces a denser, less permeable concrete; 2) when used as a cement replacement it reduces total alkali content by reducing the Portland cement; and 3) alkalis react with fly ash instead of reactive silica aggregates. Class F fly ashes are probably more effective than Class C fly ashes because of their higher silica content, which can react with alkalis.

As stated above, there exist, as well as fly ash, coarse ash particles, referred to as bottom ash or slag. These bottom ashes separate from lighter fly ashes as the ashes fall to the bottom of the combustion chamber. The lighter fly ash remains suspended in the flue gas. Bottom ash or slag has not been used for concrete and is deemed “off-spec”. The chemical composition of bottom ash is like the fly ash though it generally contains greater amount of carbon. Bottom ashes are not the tiny glassy particles but, rather, have angular particles with a very porous surface texture looking like volcanic lava. Bottom ash comprises up to 25% of the total ash while the fly ash forms the remaining 75%. One of the most common uses for bottom ash is as structural fill. But, because of its chemical makeup and because of its larger sized particles and their non-spherical shapes, there has been no conventional use of bottom ash to augment admixtures of Portland cement concrete.

Because the addition of fly ash to Portland cement improves the workability and pumpability of the resulting concrete, increases its density, increases its ultimate strength and improves the resulting concrete's resistance to chlorine and sulfate attack, fly ash demand has risen to exceed its supply. Addition of fly ash makes Portland cement more effective, reacting with excess portlandite making it unavailable to react with sulfate or carbon dioxide and greatly shrinks the size of the pores in the concrete. Fly ash displaces its equivalent weight in Portland cement which at $70-$80 per ton proves to be the most expensive ingredient in concrete. The lower price of high-quality fly ash pozzolan and its desirability as an additive makes it an especially attractive ingredient in concrete. But, the attractive nature of fly ash has caused its price to rise in areas where fly ash is less available and prices of $25-$30 per ton are not uncommon.

As stated above, fly ash is declining in both quantity and quality. Much of this decline can legitimately be blamed on NOx control. Low NOx burners result in greater unburnt carbon content and, thus, increased loss on ignition (LOI). Changing from a hot oxygen-rich flame to a cooler fuel-rich flame without substantially altering the geometry of the combustion chamber must result in higher LOIs. This increase is often enough to push quantities of marginal fly ash beyond the C-618 specification for pozzolan. So, like the bottom ash, this carbon-rich fly ash is also considered to be “off spec.”

Transportation to areas where fly ash is less available is not a solution. Fly ash is consumed and marketed regionally. It is a bulky, heavy material which attribute makes it very expensive to move. It is not unlike limestone in its economics. For example, it costs between $0.10 and $0.13/ton/mile to transport fly ash by pneumatically loaded trailer truck. Thus, there is a limit as to how far it can be economically moved in today's market. It can go further by rail or barge, but loadout facilities and rail car availability can provide serious complications. There is an increasing spot shortage of production coal ash supplies in some regions of North America with the replacement of coal in domestic energy systems.

Coal plant closures are impacting haulage distances and the market price structures create new opportunities as the United States is currently a net importer of coal ash, to spite the billions of tons of waste ash in storage. When the variations of the building industry's activities and subsequent demand are added in, it is not at all difficult to understand how regional shortages of quality pozzolan can develop. To the extent that bottom ash and out of specification fly ash, which is more plentiful, can supply material into this shortfall, the demand for resulting concrete can be met.

The need exists for a method of processing “off spec.” ash that optimizes the material for pozzolanic reaction securing for users of such ash, to duplicate the benefits of “in spec.” fly ash for use in conjunction with Portland cement in concrete.

### SUMMARY

The present invention may be embodied as method for exploiting bottom ash as a pozzolanic material in Portland cement concrete. The method includes improving the quality of bottom ash in grinding the bottom ash. Grinding or pulverizing bottom ash converts the resulting particle of bottom ash from large, porous, and irregular shapes to fine granular ash by reducing the particle size as well as eliminating its porosity. The method produces a fine granular ash which when processed with suitable additives can be used as a good pozzolanic material.

A system and method for amending out of specification ash to a suitable amended addition material to a concrete mixture includes analyzing a unit quantity of out of specification ash to determine concentrations of each of calcium oxide, sulfur trioxide, and unburnt carbon. The out of specification ash is sieved to determine fineness. Based upon measured concentrations and determined fineness, a mixture is designed to add materials in design-determined quantities of each of quicklime, kaolin, masonry silica blend, manufactured sand and in specification fly ash. By adding each of the quicklime, kaolin, masonry silica blend, manufactured sand and in specification fly ash in each of the design-determined quantities, creating an amalgamated blend of added materials to form an amended addition material. The amended addition material is substituted for a selected quantity of Portland cement in the concrete mixture.

Having as its object, the pulverization and mixing by pulverizing means of off spec ash and silicon quartz particles the inventive process yields an admixture having a very small particle size and a proper chemical balance to serve as a highly reactive pozzolan. The resulting admixture will react with the calcium hydroxide (Ca(OH)₂) as is produced when cement hydrates. The small particle size means that the particles fill the spaces between the cement grains, improving the rheology of the mix and reducing the voids in the concrete. The ‘pozzolanic reaction’ with the Ca(OH)₂ increases the amount of calcium silicate hydrates (CSH) produced, which intensifies the bond to the aggregates and improves the homogeneity of the mix, reducing porosity and permeability and increasing the strength.

### BRIEF DESCRIPTION OF THE DRAWINGS

FIG. 1A depicts a table composed and grouped for explaining the chemical constraints set out in ASTM C618;

FIG. 1B depicts a table composed and grouped for explaining the physical constraints set out in ASTM C618;

FIGS. 2A, 2B and 2C comprises three photographs which, in turn, show the appearance of cement, slag, and fly ash, and their specific surface areas are 342, 505, and 935 m²/kg, respectively;

FIG. 3 summarizes for non-limiting exemplary mixes by the inventive method;

FIG. 4 is a flow chart for preparing amalgamated blend of out of specification fly ash with added materials such as quicklime, kaolin, masonry silica blend, manufactured sand and in specification fly ash; and

FIG. 5 is a schematic representation that depicts a system for effecting the method of preparing amalgamated blend of out of specification fly ash with added materials such as quicklime, kaolin, masonry silica blend, manufactured sand and in specification fly ash.

### DETAILED DESCRIPTION

As stated in the Background set forth above, off-spec ash comprises many of the same elements as approved in-spec ash but in different proportions and generally of a coarser consistency. The inventive process exploits off-spec ash as containing many of the constituent chemicals of the more effective pozzolans but in distinct proportions. Combining off-spec ash with selected materials under the inventive method effects a chemical dissolution in counteracting the impurities such as Silica SiO₂, Sulfur Trioxide SO₃. In addition, recombining elements by the inventive means produces a positive byproduct introducing ferric oxide (Fe₂O₃), in minute quantities. Small quantities of ferric oxide (up to approximately 20%) improve the compressive strength of concrete but decreases its workability. In the small quantities resulting from the inventive method, the presence of ferric oxide is such quantities promotes nucleation (concrete-creation) and does not adversely affecting the entrained air, such adverse effects could include creating additional air voids, and ultimately not allowing the “curing” process to evolve uniformly.

Fly ash is a pozzolanic material. It is a finely-divided amorphous alumino-silicate with varying amounts of calcium, which when mixed with Portland cement and water, will react with the calcium hydroxide released by the hydration of Portland cement to produce various calcium-silicate hydrates (C—S—H) and calcium-aluminate hydrates. Some fly ashes with higher amounts of calcium will also display cementitious behavior by reacting with water to produce hydrates in the absence of a source of calcium hydroxide. These pozzolanic reactions are beneficial to the concrete in that they increase the quantity of the cementitious binder phase (C—S—H) and, to a lesser extent, calcium-aluminate hydrates, improving the long-term strength and reducing the permeability of the system. Both mechanisms enhance the durability of the concrete.

The performance of fly ash in concrete is strongly influenced by its physical, mineralogical and chemical properties. Fly ash consists of silt-sized particles which are generally spherical, typically ranging in size between 10 and 100 microns. In contrast to the heavier and jagged bottom ash counterpart particles, these small glass spheres improve the fluidity and workability of fresh concrete. Fineness is one of the important properties contributing to the pozzolanic reactivity of fly ash. Because, fly ash is typically finer than either of Portland cement or lime, fly ash formed in such small glass spheres lends the ash a fluidity to improve the workability of the fresh concrete to which it is added. Fineness is one of the important properties contributing to the pozzolanic reactivity of fly ash.

Fineness changes the geometry of individual particles and dictates the action of the ash as a pozzolan in that ash of a coarser gradation can result in a less reactive ash and because of incomplete combustion could contain a higher unburned carbon content. Limits on fineness are addressed in the concrete-relative ASTM standards and state transportation department specifications. Fly ash can be processed by screening or air classification to improve its fineness and reactivity. Geometry dictates that the finer the granules, the more surface area per unit volume is exposed to the chemical environment for reaction when the granules are mixed into the concrete mixture.

In conventional commercial practice, the incorporation of in-spec fly ash is limited in a range from 15% to 20% by mass of the total cementitious material, in embodiments of the instant invention, however, cement is replaced by the incorporated out-of-specification (“OSFA”) up to 35% of the total cementitious material. In conventional use of in-spec fly ash, the 20%-demonstrates a beneficial effect on the workability and cost of concrete. But, in conventional construction, the limitation may not be enough to sufficiently modify the mix to improve the resulting concrete against sulfate attack and to improve its durability. It is known that the substitution of conventional in-spec fly ash at volumes beyond 25% and even up to 35% has a somewhat negative impact on early strength and longer initial setting time due to the low reactivity of fly ash in general terms.

The calcium content of the fly ash is perhaps the best indicator of how the fly ash will behave in concrete, although other compounds such as the alkalis (Na₂O and K₂O), carbon (usually measured as LOI), and sulfate (SO₃) can also affect the performance of the fly ash. Low-calcium fly ashes (<8% CaO) are invariably produced from anthracite or bituminous coals and are predominantly composed of alumino-silicate glasses with varying amounts of crystalline quartz, mullite, hematite and magnetite. These crystalline phases are essentially inert in concrete and the glass requires a source of alkali or lime (for example, Ca(OH)₂) to react and form cementitious hydrates. Such fly ashes are pozzolanic and display no significant hydraulic behavior. High-calcium fly ashes (>20% CaO, Class C) may be produced from lignite or sub-bituminous coals and are comprised of calcium-alumino-silicate glass and a wide variety of crystalline phases in addition to those found in low-calcium fly ash.

The instant invention exploits a mixture of natural and artificial sands to balance impurities present in the out of specification fly ash to reach an optimum chemical makeup. In embodiments of the invention, the artificial sand is selected to be crushed recycled concrete pulverized to exploit angular fracture lines allowing a stronger matrix structure. In further embodiments of the invention, the artificial sand is crushed from recycled concrete pulverized to a very fine and angular particulate. To condition out of specification fly and bottom ash to be usable (as demonstrated in 28-day core testing), the instant invention relies upon selection of specific forms of the crystalline silicate component SiO₂ the sand provides. The instant invention includes the use of such silicate to “dilute” those prevalent compounds present in the off-specification fly and bottom ash to bring the resulting amalgam into an optimum mixture in accord with intended use. This added silicate is preferably a mixture (according to the inventive procedure) wherein as much as 70% constitutes clean washed masonry sand conforming to ASTM 2.3-3.1 and the remaining 30% or more is a careful clean/washed harp-screened man-sand produced from crushed quartz or crushed concrete, (infused at net 0.2 moisture content) which has proven to be of twofold advantage.

The first of these advantages exploits man-sand to achieve the reduction of unusually high concentrations of sulfur trioxide levels, SO₃ (a common ingredient that, by its presence in elevated concentrations causes the ash product to be classed as off-specification). In its proper concentration, the 5% maximum in the conventional specification acknowledges the need for SO₃ as its presence in the resulting mixture enhances the early age compressive strengths of mortar and concrete. Upon examination of test cores of the inventive mixture which have been found to include cement grains which have contracted dramatically (by means of dissolution); when exposed to water, sand and small rock particles, especially those of reduced particle size, release calcium and silicon ions. These ions spread throughout the mixture reach a critical concentration in solution. The presence of artificial sand (made by crushing rocks or concrete to produce fine particulate or “man-sand”) and its silicate facilitates the eventual precipitation out of solution which then forms the cement coating (cation) film around the aggregate particles and yields a binder having superior mechanical, physical and durability attributes compared those of conventional geopolymer concretes made using conventional ashes containing Alumino-Silicates. The inventive concentrations promote the production of the above-described cation film.

At low Ca/Si, cations (Ca₂⁺, Na⁺) are present in the diffuse layer compensating the negative surface charge of C—S—H and those cations are easily exchangeable. The presence of alkali hydroxide increases the pH and leads to the replacement of Ca₂⁺ present in the diffuse layer by Na+. The presence of KOH and NaOH leads also to a somewhat more negative zeta potential, due to the deprotonation of silanol groups, which results in a moderate increase of the measured effective cation exchange capacity (“CEC”). The presence of NaOH increases in addition the ionic strength, which reduces the double layer thickness around the C—S—H particles.

The value of the effective CEC of C—S—His only weakly influenced by the pH value but strongly decreases due to the presence of specifically bound Ca₂⁺ at high Ca/Si. In hydrated Portland cement, where C—S—H shows a Ca/Si ratio of 1.5-2.0, the capacity of C—S—H to bind cations is expected to be small. In contrast, blended cements containing silica-rich supplementary cementitious materials, which results in C—S—H with lower Ca/Si, can be expected to bind more cations. C—S—H gel has a pore structure with multi-dimension, and the diffusion of water and ions in the C—S—H gel determines the chemical and physical properties of cementitious materials, such as strength, creep and shrinkage. Silicate anion is the basic unit of C—S—H gel. In the structure of silicate anion, the tetrahedral units [SiO₄] are linked together by sharing oxygen atoms to form Si—O—Si bonds.

Among sources of silicate for amending off-spec ash, virgin stone crushing does generate its own shavings and does yield fair results. Those shavings generated through pulverization of concrete preferably by known crush methods by any of cone, impact or jaw crushers. In conventional process, the shaving by-product of crushing concrete or even virgin stone would generally be disposed of as “refuse.” In the conventional context, use was limited to select utility embedment encasements and landscape top dressing. The inventive method, however, exploits the extremely fine sieve counts of such refuse which makes it suitably well adapted for inventive method. Because the fineness of this crushed manufactured sand accounts for the preference of embodiments exploiting a vertical shaft impactor as the resulting sand exhibits a multi-fracture microstructure presenting smooth faces. Because the concrete from which the artificial sand is generated by crushing has previously been designed for strength, the inherent oxide levels in the resulting man sand are low. Without these oxides, there are no ions to form pozzolanic bonds to augment the matrix the hydraulic reactions form.

With its fracture surfaces being smooth, the man sand inherently can conform more easily to such void spaces as the cementitious processes produce and therefor packs cleanly into the resulting ceramic matrix allowing more complete consolidation. This quality of crushed concrete as aggregate is particularly advantageous while forming a cation in the nucleation phase while simultaneously coagulating. By way of contrast natural sand only yields a coarser product incapable of similar nucleation. In addition, the fineness of the crushed concrete allows for greater efficiency of consolidation and, thereby, results in less severe crack propagation; rather than to force the coagulation of tobermorite gels while using virgin sands which possess no such semi-cementitious nor consolidation qualities, resulting in wide-spread propagation of cracks. Also, because natural sand present unequal ranges of carbon levels, exploiting ashes of various chemical make ups by blending with sands of various make ups lends no known levels, making it difficult to control the quality of the resulting mix. The process requires an assay of the OSFA to determine the specific amending silicates necessary to bring the ash into chemical compliance with the design mix desired. Generally, it is an object of the inventive method to keep the manufactured sands at a proportion of 55% relative to natural sand. Thus, where clean washed natural silicate sand is blended with artificial sand to replace OPC those ratios would generally not exceed 37% for crushed concrete artificial sand and 24% natural stone crushed rock sand such that the remainder is supplied as clean concrete silicate washed sands.

Off-Spec fly ash has a high carbon and calcium content, which previously eliminated it from use with cementitious materials. The carbon within Off-Spec fly ash absorbs air-entraining admixtures in freshly mixed concrete, making it very difficult to control entrained air. To the end of producing a high-performance concrete mix, the silicates are added to change the chemical content of the resulting mixture. These sands are mechanically blended wherein the natural washed mined silicate concrete sand is present in a ratio of between 50-55% depending upon the make-up of that natural sand, concrete” pulverized artificial sand comprising shavings which are clean and washed to contribute in a ratio of 30-37% or if using natural stone shavings “man-sand” present in no more than 24% though augmented with filtered quick lime CaO (often from kiln dust) at a ratio of 8-7% and including “clean” Kaolinite pulp at a ratio of 3-4%. The intergrinding of the raw mix is more advantageous than separate grinding as far as the reactivity is concerned.

The deficiencies in OSFA are that (1) OSFA has limited reactivity for hydraulic and pozzolanic reactions, because OSFA has a limited percentage of reactive oxides such as CaO, SiO₂, and Al₂O₃ while the reactivity is essential for development of concrete; (2) typically has a high content of carbon that absorbs air entrained as a chemical admixture that is used in conventional concrete to improve air content for long-term durability; absorption of an air entraining admixtures compromises the durability; (3) contains an over-abundance of unburnt carbon thereby imparting a low density to the resulting mix and such carbon can float to the casting surface during the casting and finishing processes, producing heterogenous microstructures and an aesthetically undesired surface. Thus, once the method accomplishes an assay of the OSFA to know its make-up, the suitable proportions of above-described silicates are selected to “dilute” the over-abundance of unburnt carbon and to augment the reactive oxides.

The benefits garnered by benefits from using fly ash in concrete:
a. The Calcium Silicate Hydrate (CSH) that is produced from the pozzolanic reaction has a lower CaO: SiO₂ (C/S) ratio than the CSH that is produced when the calcium silicates from the OPC react with water. This CSH gel with a lower C/S has a greater capacity to entrap alkalies and reduce the pH of the concrete pore fluid.
b. The pozzolanic reaction consumes calcium hydroxide (CH) which is an abundant hydration product in concrete. The gel that forms in a paste with reduced amounts of calcium hydroxide may have lower swelling characteristics.
c. The pozzolanic reaction produces a denser paste by reducing the amount of calcium hydroxide and producing additional C—S—H gel. The additional C—S—H is formed at the paste aggregate interface. Alkali-Silica Reaction (ASR) is a neutralization reaction that occurs between an acid (silicic acid) and a basic pH pore solution (Calcium Oxide, Potassium Oxide, and Sodium Oxide). ASR causes deleterious expansion within concrete, which can cause durability issues decreasing the life span of concrete. This C—S—H gel reduces the mobility of ions and possibly slows the ASR reaction rate. It also reduces the concrete's permeability which helps prevent external moisture and alkalis from entering the concrete.

The above-described inventive blend also is advantageous at the stage of nucleation (an initial reaction that occurs during mixing of concrete) in that the nucleation at those proportions enhances the C₂S and C₃S reactions to yield more tobermorite gel C₃S₂H₃ expressed in the above shorthand. Tobermorite is a calcium silicate hydrate mineral with chemical formula: Ca₅Si₆O₁₆(OH)₂·4H₂O or Ca₅Si₆(O,OH)₁₈·5H₂O. With the man-sand-supplied silicates, all pozzolanic reactions are specifically concrete while the hydration actions will be slower than as occurs in conventional concrete mixtures with the inclusion of fly ash (either of Class C or F). Using the inventive procedure, the measured early strength yet will be approximately equal and will exceed that measured in long-term strength. By generating the enhanced hydrate coatings and cation as these are produced by fewer than the volume of normal sphericals which occur in conventional concrete composed of class C or class F fly ash alone.

A second advantage in using the out of specification ash along with the suitable volume of silicate in the form of man-sand is exhibited in the cycle of dissolution, diffusion, and precipitation in the formation of concrete. Concrete, itself, is a non-homogenous, brittle material and failure in both compression and tension occurs by crack propagation within the matrix of its base constituents, now this is derived from the microscopic voids (or stress concentrators) inherently in every mix. Within the matrix, the non-spherical nature of the OSFA is such that it cannot be packed into a given volume without a significant number of microscopic voids. Angular granules of silicate such as those that make up man-sand allow a tighter packing of the matrix filling, at least partially, the voids that might occur in the absence of man-sand.

The advantages in reduction of such space cannot be achieved by simply adjusting the conventional elements of concrete, i.e. cement, ash, sand, aggregate, and water. OPC can be replaced more than 40% by a pulverized volume of out-of-specification fly and bottom ash because the inventively blended ash can exploit the reduced base particle size to yield a finished concrete without any deleterious effects. In the inventive procedure, the pulverized granules which will form the tobermorite gel which will form from the combined, pulverized sand, water, and cement and will flow to fill the voids at least to an extent greater than might occur in conventional concrete. It is at hydration where the concrete is beginning to “set” wherein the pulverized OSFA and man-sand mixture sets up to form a tighter matrix in that the triply fractured granules adhere and conform to eliminate, to a far greater extent, some of the void spaces which occur in the conventional matrix and in setting up in the inventive matrix results in less crack propagation in the finished product and a more rigid finished product, able to withstand greater loads while using the same material constituent values.

As stated above, in the inventive method, the silicate is important not only for its chemical makeup but also for the cubic geometry of particles. Many sands and all the highest quality sand deposits are made of quartz enjoy this cubic geometry. Quartz is a hard, crystalline mineral composed of silica having an overall chemical formula of SiO₂. Quartz is the second most abundant mineral in Earth's continental crust, behind feldspar, comprising an estimated 35% of all rocks. A mineral is a naturally occurring inorganic element or compound having a periodically repeating arrangement of atoms and characteristic chemical composition, resulting in distinctive physical properties. The crystal structure controls physical properties of minerals. In quartz each oxygen atom is shared between two silicon atoms in a tight three-dimensional framework giving rise to a mineral that is very stable both chemically and mechanically.

Sand, either naturally occurring or that produced by crushing concrete, will provide silicate as a precursor to the concrete mix. With control of the size of the precursor's particles, it is possible to control the self-foaming effect (reaction of compounds present in precursor with alkali) through allowing/forbidding the dissolution. With control of the particle's size of precursor, it is possible also to control packing of the particles, i.e. smaller the particles the closer the packing, which results in higher mechanical strength (experiment from precursor's particles between 1 mm and 2 mm). With particles' size, it is also possible to control the size of the voids which allow the escape of gasses or get filled with dissolved material in the slurry from which concrete forms.

The self-foaming effect showed high impact only in the fraction below 1 mm, i.e. in the smallest fraction. Diffusion of dissolved precursor's compounds and their distribution have an important impact in “polymerization” in alkali activated synthesis and should be limited to the local surroundings (diffusion limited “aggregation”) to gain faster solidification (property desired in the industry). This can be gained with the addition of the lowest amount of liquid possible to the precursor, i.e. to have highest possible viscosity of the slurry (drying needs less time and diffusion is limited with high viscosity). Compressive and bending strength of alkali activated material showed unique dependence on refractory material's particle's fraction where up to certain fraction (down to 1 mm) such that the smaller the fraction the higher the mechanical strength. With the smaller the particles, they each present a bigger the reactive surface area in comparison to the volume thus allow a higher reaction rate of the precursor and more reacted final material, which would make a cured concrete with better mechanical strength if alkali activation would not cause precursor to foam, i.e. when alkali activating precursor has tendency to self-foam, the mechanical strength depends on the amount of reacted material and porous structure in the reacted material. If gases can be released from the formed concrete through the voids between particles in a larger fraction of the precursor, they do not affect the mechanical strength as much as when they destroy the compactness of the formed concrete leaving it with lower density, and lower compressive and bending strength. Highest mechanical strength had a sample with fraction 1 mm<x<2 mm, which had the best close packing of not-reacted precursor's particles and still was not influenced by foaming effect.

Artificial sand, also called crushed sand, mechanical sand, manmade sand, or “man sand” and is crushed from rocks, mine tailings or industrial waste granules with a particle size of less than 4.75 mm. Man sand is derived by mechanical crushing and sieving of these source rocks in a process known as beneficiation. “Beneficiation” is the technical term describing the industrial process of mechanically separating minerals from each other. No chemical changes to the minerals are made at this point in the mining process. In the preferred embodiments of the invention, crushed concrete is used to provide some part of the man sand that makes up the final inventive mix.

Artificial or man-sand may be derived from concrete is, generally, obtained from any of several scenarios. Two exemplary but not limiting examples are as follows:
a. Method 1—crushing concrete to produce coarse and fine fractions i.e. concrete wastes are primarily crushed in a jaw crusher and sieved at 4.75 and 19 mm; the fraction greater than 19 mm is then submitted to secondary and tertiary crushing in a cone crusher and sieved again to obtain the coarse fractions (19-4.75 mm); material passing at 4.75 mm is then washed to obtain the fine aggregates (4.57-0.15 mm); material below 0.15 mm was not mentioned or characterized.
b. Method 2—crushing concrete to obtain fine aggregates by successive comminution stages wherein concrete wastes are crushed primarily in a jaw crusher in a closed circuit with a 50 mm screen, then crushed again in an open circuit with a secondary jaw crusher, and then with a tertiary crushing in a roll crusher configured in a closed circuit with a double deck screen with 4.75 and 0.60 mm apertures; the fraction passing at 0.60 mm is, then, washed in a wheeled sand washer to remove the fraction below 0.15 mm and then combined with a sieved fraction of 4.75-0.60 mm to obtain the final recycled fine aggregate; material below 0.15 mm was not mentioned or characterized.

By way of better explanation, while each of the two methods produces fine recycled aggregates by crushing concrete wastes from the same origin; the water absorption of aggregate produced by the second method was 25% lower than that by the first method (6.6 and 8.9%, respectively). The particles produced by the first method are rougher in shape and more angular than those produced by the second method due to the second method's repeated crushing steps and the lack of coarse aggregate used in the second method of production, such that it contains a higher percentage of natural aggregate. As indicated, while either will work to produce a man sand suitable for the instant invention, contrasting aggregate produced by these methods demonstrates clearly that the crushing process significantly influences the quality of the resulting fine recycled aggregates.

The particles by the first method, being rougher in shape and more angular than those produced by the second method, are those of the presently less-preferred embodiment. Recycling plants around the world focus on the production of coarse recycled aggregates, while the sand fraction is involuntarily generated as a by-product (in a few cases), but mostly as tailing. These tailings are not of great use as a natural sand substitute because its composition is mostly fragile particle edges that broke off during handling or demolition as well as in the grinding processes. These sliver-like shavings are not as readily effective in the formed matrix. The jaw crusher's aperture affects the proportions of coarse and fine fractions but do not make a notable change in particle size distribution of the fine fraction, when the process is set for producing coarse aggregates.

Either mineral quartz or recycled concrete are crushed to produce an artificial sand of a specified fineness and regular particle size known as “man sand.” The presently preferred embodiment of the invention exploits man sand produced by use of vertical shaft impactors (“VSI”). VSI crushers use a high-speed rotor and anvils for impact crushing rather than compression force to provide the energy needed for size reduction. The production of sand from crushed stone (artificial sand) has been conducted for over a decade using VSI crushers or rotor impact mills. These machines consist of a rotor revolving at high speed that throws centrifugally the material into the crushing chamber, where the comminution occurs by rock-on-rock impact, attrition and abrasion. The main advantage of these systems is the ability to improve the roundness and sphericity of the particles, contributing to a better aggregate morphology.

The quartz to be crushed is fed into the center of an open or closed rotor. The rotor rotates at high rpm, accelerating the feed and throwing it with high energy into the crushing chamber. In a VSI, material is accelerated by centrifugal force by a rotor against the outer anvil ring, it then fractures and breaks along natural faults throughout the rock or minerals. When the material hits the anvil ring assembly, it shatters, and then the cubical shaped product falls through the opening between the rotor and the anvil and down to the conveyor below. The resulting product is generally of a consistent cubical shape, making it angular with sharp edges. Due to cleavage breaking of the particle, the resulting granule surface is smooth. Thus, manufacturing the man sand by action of the VSI is employed in the presently preferred embodiment. In contrast, fine aggregates manufactured by compression crushing (jaw crusher, cone crusher, roll crusher, hammer mill) are flaky and more irregularly angular in shape and with a resulting surface texture that is very rough.

The crushing characteristics of hardened concrete produced by VSI are like those of natural rock and are not significantly affected by the grade or quality of the original concrete. So long as the softer, more porous hydrated cement paste has been previously segregated, recycled concrete aggregates (RCA) produced from all but the poorest quality original concrete can be expected to pass the same tests required of conventional aggregates.

Recycled concrete aggregates contain not only the original aggregates, but also hydrated cement paste. This paste reduces the specific gravity and increases the porosity compared to similar virgin aggregates. Higher porosity when present in RCA leads to a higher absorption in the final mix. Because where hydrated concrete paste is not previously removed, i.e. where RCA contains mortar from the original concrete, it is more porous and absorptive than many natural aggregates. The presence of the hydrated concrete paste does affect the quality of final mix and must be removed. For example, recycled coarse aggregate including such concrete paste generally has a water absorption of 5% to 6%; and recycled fine aggregate absorption was 9% to 10%. In contrast, natural aggregate typically has an absorption of 1% to 2%. For this reason, the design of the resulting mix must include this consideration to fine tune the attributes of the mix if the paste cannot be earlier removed. In recycled aggregates, the most common property to be evaluated is density due to the high porosity of hardened cement paste in contrast with low porous virgin aggregates. On the other hand, when suitably selected, the compressive strength and splitting tensile strength of the concrete are not substantially affected using recycled aggregate.

Another advantage of the inventive triply fractured mixture of OSFA and man-made sand is that the resulting mix can concentrate the heat the exothermic reaction produces. By concentrating the energy during hydration, the temperature within the concrete matrix will be raised beyond that achieved in the conventional reaction. Because of the more tightly formed matrix and the greater heat produced in hydration, the finished concrete product is of an equal or higher density than that formed by conventional means. The resulting concrete according to the inventive procedure is an extremely complicated material with a fairly disorganized atomic structure dictated by the physical configuration of the triply fractured granules in the resulting matrix.

By its nature, OSFA, either bottom or fly ash, is not uniformly out-of-specification. No procedure to remediate ash ought to progress without knowledge of the raw material to be used. Thus, the first step in the inventive method is to ascertain the chemical makeup of the OSFA. By way of example, where coal for burning to ash is sourced from distinct geographic locations, the chemical makeup of the coal varies. Combustion does not make the ash any more uniform than was the coal that produced the ash. For that reason, ash the inventive method exploits, optimally, is to be analyzed in order finely ascertain what volumes of man-made sand to be added to suitably dilute elements within the ash to make the material usable to produce an inventive concrete mix.

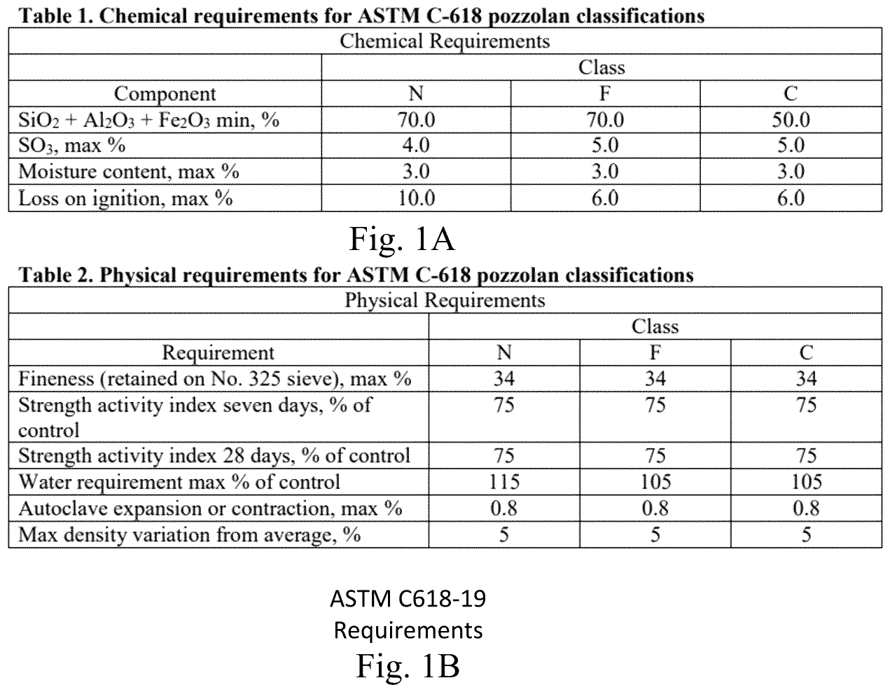

Pulverization of the OSFA to create small spheroid granules and includes the screening the granularity of the pulverized ash. ASTM C136-84a is entitled, “Standard Method for Sieve Analysis of Fine and Coarse Aggregates.” C₁₃₆-84a dictates the screening as to particle size distribution. A sieve analysis (or gradation test) is a practice or procedure used to assess the particle size distribution (also called gradation) of a granular material by allowing the material to pass through a series of sieves of progressively smaller mesh size and weighing the amount of material that is stopped by each sieve as a fraction of the whole mass. The size distribution is often of critical importance to the way the material performs in use. Being such a simple technique of particle sizing, it is probably the most common. FIG. 1A depicts a table composed and grouped for explaining the chemical constraints set out in ASTM C618; FIG. 1B depicts a table composed and grouped for explaining the physical constraints set out in ASTM C618.

The formation of tobermorite is strongly dependent on the source, composition, and particle size of the aluminosilicates. The properties of alkali-activated materials (AAM) depend on the chemical composition of the source material (precursor). Aluminosilicate systems based on ash with low calcium content require a heat treatment curing cycle, which makes these materials well-suited for prefabrication and precast manufacturing processes, but they jeopardize their sustainable character because they require a significant amount of thermal energy to set and harden. Because the ash, itself, is the product of a high-heat process, adequate calcium is one of the attributes sought in the out of specification ash used as material in the inventive process. Other chemical attributes are also necessarily considered in the process.

The inventive method does further amend the blend, optionally, with the addition of clay in the form of kaolin. Kaolin was selected as its reactivity to alkaline activation at ambient temperature is negligible, and hence it does not interfere in the reaction sequence. Kaolinite is generally unreactive to alkali attack at ambient temperature. However, the addition of clay may affect chemical reactions as occurs in clay-cement mix. Besides, kaolin represents a wide class of clays encountered in engineering projects. Kaolin, or, as known in the vernacular, China clay, is nearly white in color and is distinguished from other industrial clays based on its fine particle size and pure coloring. The primary constituent in kaolin is the mineral kaolinite, a hydrous aluminum silicate formed by the decomposition of minerals such as feldspar. Thus, kaolinite is used to add the aluminum silicate to supplement that contained in the OSFA.

For a system made to include lime and kaolin, its pozzolanic activity depends on the dissolution of kaolinite which provides the only source of aluminum and silicon. That kaolinite possesses a stable crystalline mineral structure making it hard to dissolve, such that this mineral structure determines the slow reactivity of that system. By contrast, high-calcium fly ash contains reactive phases i.e. calcium-rich phases primarily and to a small extent its vitreous phase (for high-calcium fly ash) thermodynamically less stable than kaolinite and, therefore, easier to dissolve. That is why reaction sequences are faster for both high calcium fly ash systems.

As stated above, the analyzed OSFA is pulverized in an impact crusher. Pulverizing and classification of dry materials is conventionally by using hammer-screen mills, impact attrition mills, ball mills, pin mills and others outfitted with internal classifiers that separate the coarse and the fine particle fractions. The air classifying mill is popular equipment for pulverizing and separating particulate material into selectively sized particles. In general, air classifying mills can be classified into two types; single- and dual-drive.

Dual-drive mills can pulverize a wide variety of materials and allow the operator to control the particle size simply by drive speed adjustment. Typically, dual-drive air classifying mills comprise a housing containing a pulverizing disk and a particle classifier, wherein each of the pulverizing disk and the classifier are controlled by a separate drive. One drive adjusts the classifier speed to control the particle size while the second drive controls the pulverizing disk speed, which is adjusted depending on the hardness and toughness of the particulate material. Because the speeds of the classifier and the pulverizing disk are each controlled by a separate drive, they can be adjusted independently.

Single-drive mills are of more simple construction, less expensive, and more rugged because both the pulverizing disk and particle classifier are driven by the same drive. As such, the operator has limited control over particle size, and versatility with respect to material hardness is very limited. Consequently, there is less control of particle size and the type of particulate material that can be pulverized is relatively limited. However, given the single purpose to which the instant invention is directed, i.e. the pulverization and classification of OSFA, a single drive mill can also be configured to deliver a suitably pulverized OSFA.

With fineness being the primary physical characteristic of fly ash, that relates to its pozzolanic activity (and efficacy) ASTM sets the limit for a maximum amount of fly ash allowed to be retained on the 45 μm (#325) mesh on wet sieving at 34% as a quality measure. The average sizes lie in the range of 7 to 12 μm. Thus, in addition utilizing this gradation is dictated in that particles larger than 45 μm have negative effects on 30 to 90 days strengths of ceramics and Portland-fly ash mortars. At the same time, particulate is sorted by specific gravity as the specific gravity is an indicium of shape. With these factors in mind, the especially useful in its application in that crystalline silicate and amorphous silica counts are at optimum.

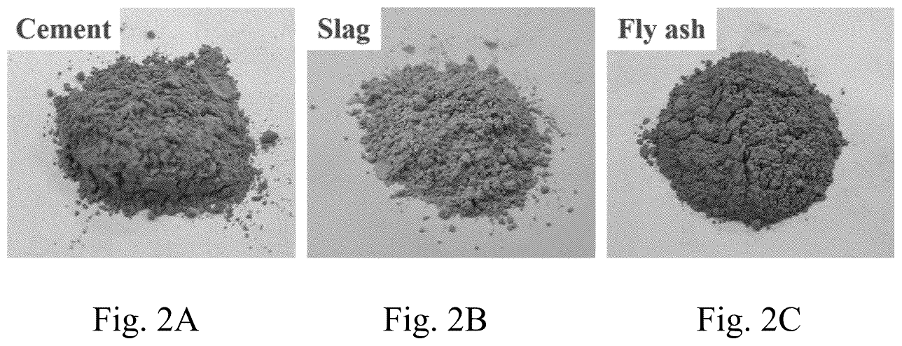

It is important to see that the intended pulverization is such as to yield smaller particles than are common in ordinary Portland cement (depicted at FIG. 2A). Even in its native form, OSFA, for example, slag, is far finer than Portland cement (depicted at FIG. 2B). Pulverization in the case of the inventive blend is to at least achieve the fineness of fly ash (depicted in FIG. 2C). In pulverization and mixing, Silicon SiO₃ from the off-spec is exchanged (cohesion) granules, and the Silicon SiO₃ from (at net-zero moisture levels) man sands are brought together in uniformity at the point of exposure in blends.

Where necessary, the pulverized OSFA can be modulated with the inclusion of crushed concrete clean silicate fines which, when added, tend, more than any other factor, to neutralize erratic sieve counts and normalize them by dilution of the off-spec fly ash to acceptable levels. The addition of clean on-spec “Class C” ash additive only acts to enhance the presence of coal particle impurities which in many off-spec ashes may be encountered at 1.3% to 1.6% and raise them to a more acceptable 2.11% to 2.13%. Because of this addition to balance, the resulting pozzolanic activity of the blend is increased as well. Thus, the resulting concrete will tend to have less crack propagation and enhance the packing of the matrix thereby eliminating common void spaces.

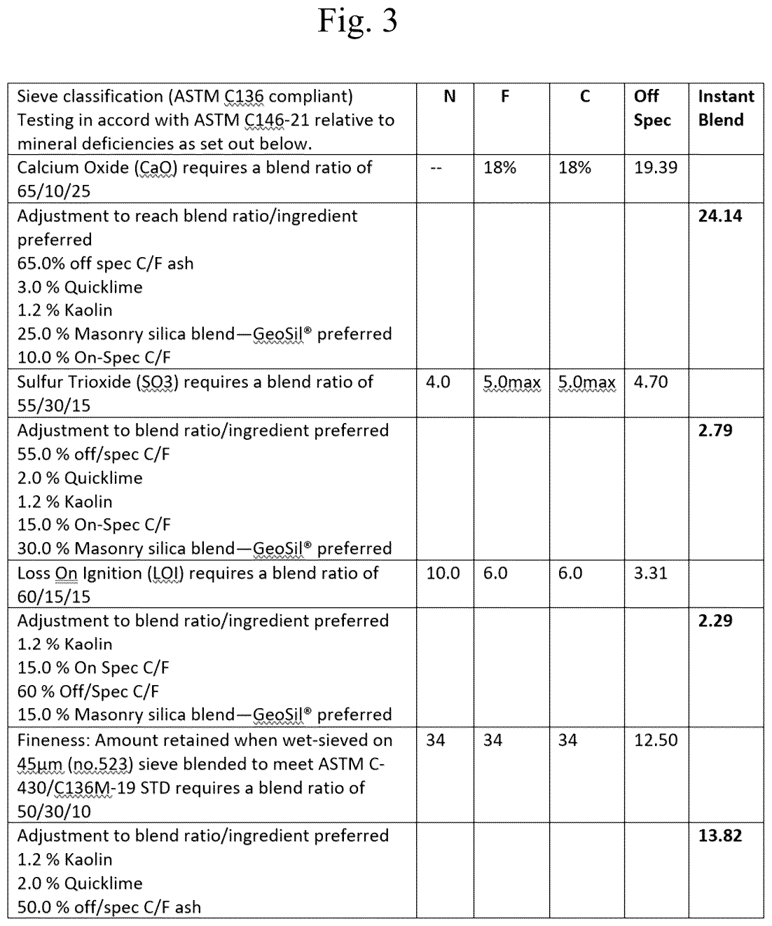

Referring to FIG. 3, several examples of the blending are set out. Starting with the predominant factor, i.e. the presence and concentration of calcium oxide (CaO) wherein the C-618 standard requires the level to be below 22%. Thus, the blend ratio is selected to provide additional calcium oxide to the resulting blend.
Example One; The provided off-spec ash does not include enough calcium oxide to reach a level of 22%. For that reason, the blend is designed to provide the shortfall. Thus, to reach that 22% level and to exceed it to a level of 24%, the constituent parts are set out:
   a. 65.0% OSFA
   b. 3.0% quicklime
   c. 1.2% kaolin
   d. 25.0% masonry silica blend sand
   e. 10.0% on-spec C/F ash
Example Two: The provided off-spec ash includes 4.70% sulfur trioxide (SO₃) wherein the target is to yield a blend having less than 3% content. Thus, to dilute the level, the blend is designed to include the following in the corresponding levels:
   a. 55.0% OSFA
   b. 2.0% quicklime
   c. 1.2% kaolin
   d. 15.0% on-spec C/F ash
   e. 30.0% masonry silica blend sand
Example Three: The provided off-spec ash includes unburnt carbon to a level that exceeds the preferred target of 2.5. In response, the blend will be designed in accord with the following specification:
   a. 60.0% OSFA
   b. 1.2% kaolin
   c. 15.0% on-spec C/F ash
   d. 15.0% masonry silica blend sand
Example Four: The provided off-spec ash is not sufficiently fine to produce the desired reactivity. In response, the blend ratio is to be configured to be of the following proportions to result in the final percentage of 13.82:
   a. 50.0% OSFA
   b. 2.0% quicklime
   c. 1.2% kaolin

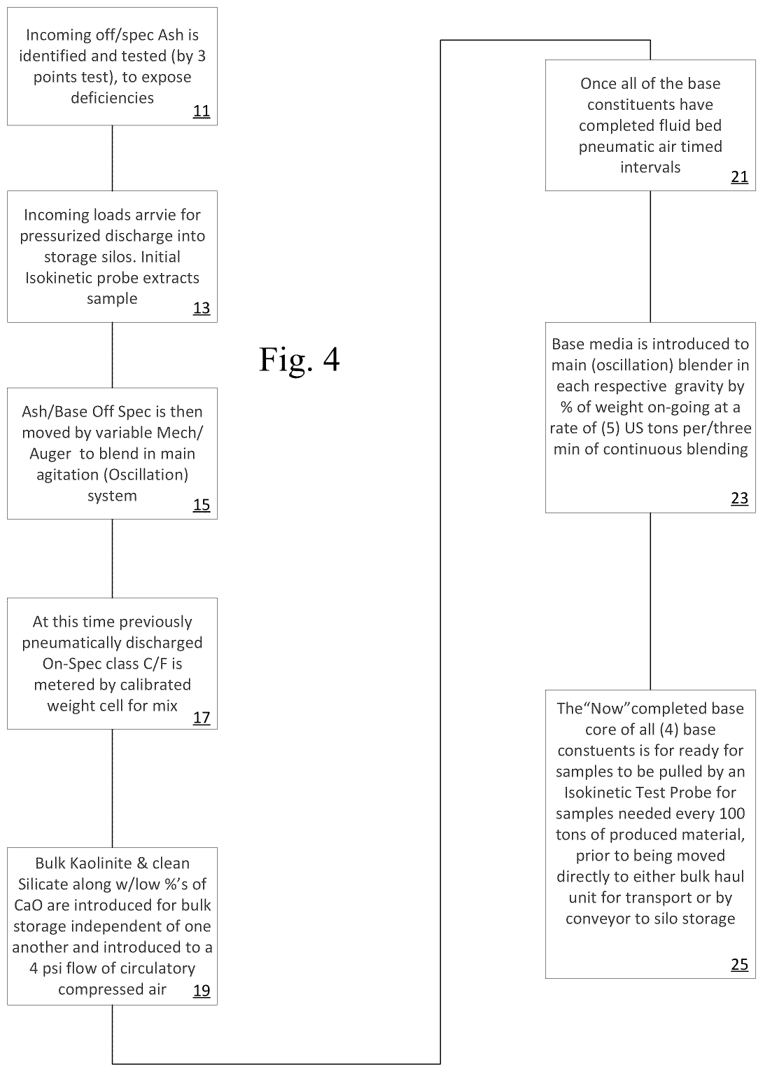

Referring now to FIG. 4, the inventive process commences at Block 11, as the provided OFSA arrives from its source, e.g. power plant, it comes in suitable transport. Generally, each load will be conveyed in a 25 ton truck or truck and trailer combination wherein the ash is carried as bulk, loose material. Specifically, such a truck or trailer includes a hermetically sealed tank is employed to preserve the purity of the materials keeping those materials free from ambient moisture or precipitation.

Optimally, at Block 13, the hermetically sealed, encapsulated 1,000 ft.³ tank pressurized to 11 psi prior to ash being off-loaded. The load is discharged from tank, preferably by umbilical hose, motivating material pneumatically to convey the same into each respective sealed storage silo. The umbilical conveys material as entrained in a flow of air making it less susceptible to internal condensation especially during inclement weather or during temperature cycles during storage. Conventional technology exists to maintain the material and to preserve it as generally anhydrous and pure.

At a Block 15, staging the material is, in the presently preferred embodiment, a computer actuates gates on silos that feed the materials into a ribbon mixer, the materials including such as OSFA; quicklime; kaolin; masonry silica blend (crushed concrete man sand blended with natural sand); and, on-spec ash, the introduction proportions are selected in accord with the blend design. Various existing technologies regularly compose mixes of various particulates such as in the case of ready mix for concrete preparation. Such conventional technologies also measure each of specific gravity, pH, relative weight, and moisture for the incoming charge of OSFA. The measured metrics relative to the incoming OSFA is used to calibrate the design of the concrete blend. Selected proportions of materials are dictated by an algorithm to keep all within designated attributes of the resulting blend.

At a Block 19, in a preferred embodiment, ultrasonic vibration is as a penetration aid as has been used with respect to granular materials since 2017. Ultrasonic conveyance with an auger is preferred because, in auguring, the relatively low friction of the flights due to vibration causes the spoil to preferentially slide up the scroll. If the reduced forces seen in sonicated penetration events could be recreated in a sonicated rotating system, then the differential friction effect that leads to elevation should be augmented and the auger should operate more effectively. In the presently preferred embodiment, a 6″ mechanized (mounted beneath the silo) auger moves off-spec ash at a rate of approximately 189.25 tons per hour (3.1 tons per minute) along a 12 linear foot auger to deliver pulverized OSFA into a sealed 550 c/f sealed compartment (hopper). An electric variable frequency motor selects a suitable speed based upon measured fineness and specific gravity of the OSFA to arrive at a rotary pugmill for mixing.

At the rotary pugmill, the measured metrics dictate a blend of the off-spec ash and masonry silicate turning in the presently preferred embodiment rotated by an electric variable frequency motor regulated to turn at an optimal rate. The added calcium oxide, silicate and kaolin clay are also added in selected proportions by augers to assure even blending at the pugmill.

At a Block 23, a fluidized bed is exploited as a bulk blending unit. A fluidized bed exploits a physical phenomenon that occurs when a solid particulate substance (usually present in a holding vessel) is suspended by jets of gas, generally air under the right conditions so suspended on the stream issued from those jets, it behaves like a fluid. Fluidized beds have many advantages as gas-solid reactors or powder handling processors because of its advantages relative to high heat and mass transfer, while maintaining temperature homogeneity, and assuring complete mixing.

In a rotating fluidized bed (RFB), particles are forced toward the rotating cylindrical air distributor due to the large centrifugal force and formed annular bed near at the air distributor. Air flows inward through the air distributor, and then particles are balanced by drag and centrifugal forces, leading to achieve uniform fluidization. Since the minimum fluidization velocity increases with an increase in the vessel rotational speed, a RFB can prevent from forming large bubbles at any higher gas velocities by controlling the rotational speed. An RFB can fluidize very fine particles, such as Geldart group-C particles, and even nano-particles, since it can impart high centrifugal force and drag force to particles, which are larger than the cohesive forces between particles.

The usual way to achieve a fluidized bed is to pump pressurized fluid such as air into the particles to suspend this uniform and mixed blend of particulate. In the presently preferred embodiment the fluidized bed has a combined bulk capacity/capability of 4,000 cubic/feet. To further enhance the mixing, pneumatic circulatory proprietary nozzles introduce a flow of air at 3 psi located in each compartment (internally) on each corner 14″ inches off bottom for maximum effect. Because this bed is closed to the ambient, it is possible to pressurize the bed. Additionally, mixing is further enhanced by exploiting a variable-frequency counterweight vibratory units mounted on the outer walls to eliminate friction there as with the ultrasonic vibratory augers.

The suitably agitated particulate is collected on cleated belt system which runs underneath the entire length of the fluidized bed which in a preferred embodiment moves at a rate of 160 ft/min. The hopper is formed to define a bottom bay door selectively opens at a predetermined to place material into mechanized bulk blending. As materials are carried down the length of the conveyor which is optionally placed at a 0% incline.

A 30-yard open top bin feeder empties into its bottom elliptical 6% incline hopper, which subsequently mechanically (by belt) carries the material to disperse at a ratio of 75 ton/hour to be deposited into the bottom entry point hopper having an 8% incline radial stacker leading to a rotary pugmill blender. The clean, washed, screened masonry sand (1.22 passing #200 sieve) is added for a final blend of material

At a Block 25, the now fully blended pulverized materials are tested for quality as against batches, in the preferred embodiment, of 55 tons. Attributes of the resulting blended materials are to test relative density, sieve counts (gradation), pH, and moisture level. Because of the electronic or computational equipment batch documentation may optionally be kept will be for a selected duration, for example 5 years, to allow further optimization of blend design based upon the strength measurements of formed concrete occurring after the concrete pour for the relevant batch.

Finally, the blended material is conveyed to a hermetically sealed silo to be stored at generally a moisture level of 3.2% and is transferred at 33 ft./min. Mechanical transfer is chose to exploit the mechanical friction to tumble the now-finished product to further dry the product dropping its innate moisture content even further to an optimal 0.09%.

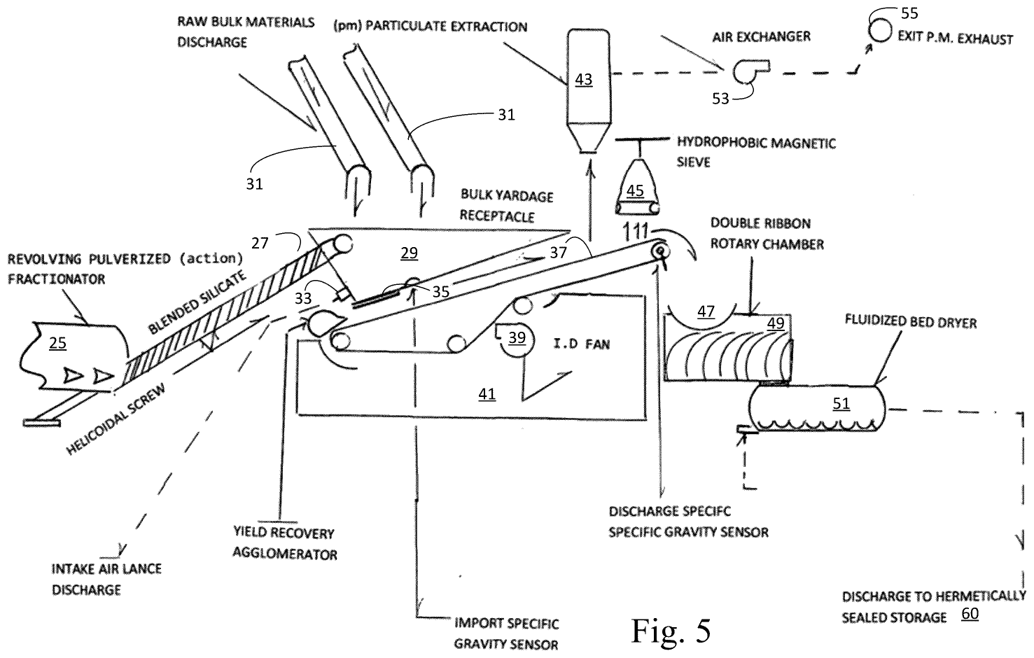

Another preferred embodiment, as depicted in FIG. 5, a revolving pugmill with a fractionator 25 pulverizes the silicate (both crushed concrete (as discussed above) and natural sand in selected proportions) to a granularity ranging in size from 0.1 to 100 μm (3.94×10⁻³ in.) with a bulk density of between 0.54 to 0.86 g/cm³ (33.71 to 53.69 lb./ft.³), generally having a specific gravity of between 2.1 to 3.0, and a specific surface area between 0.3 and 1.0 m²/g. A helicoidal screw 27 conveys the blended silicate to a bulk yardage receptacle 29.

The blend of silicate is selected as discussed above. The previously pulverized OSFA and OPC are introduced through gated (raw bulk materials discharge) hoppers 31 in specific proportion to the blended silicate to be blended in the bulk yardage receptacle 29. Further blending (not pictured) of the blended silicate may, optionally, be achieved by a revolving pugmill with a ribbon mixer.

As discussed above, it is established that particle sizes directly affect outcomes in that when the two types of sands, i.e. man-sands and natural sands are blended (though neither promote negative results in the final mix). Natural stone man-sands tend to yield elongated & flat particles (as when tested by ASTM C1252 where the measured voids could exceed 46).

As such, the contents of the bulk yardage receptacle 29 are a fine particulate and thoroughly blended admixture of OPC, OFSA, and blended silicate. This mixture is allowed to pass through a metered gate 35 to lay uniformly on a perforated chevron conveyor belt 37. A plenum 41 is pressurized by an induced draft fan 39 to generate a flow of air through the perforated chevron conveyor belt 41 to carry such fines as might be entrained up to a cyclone separator 43 based upon a cyclone further energized by a second ID fan 53.

Cyclone separators 43 work much like a centrifuge, but with a continuous feed of particulate entrained in air. In the cyclone separator 43, the particulate and entraining air is fed into a funnel-shaped chamber. The flow of air inside of the chamber creates a spiral vortex, similar to a tornado. The lighter components of this air and particulate mixture have less inertia, so it is easier for them to be influenced by the vortex and travel up it. Contrarily, larger components of particulate matter have more inertia and are not as easily influenced by the vortex. Since these larger particles have difficulty following the high-speed spiral motion of the gas and the vortex, the particles hit the inside walls of the container and drop down into a collection hopper (not shown). These chambers are shaped like an upside-down cone to promote the collection of these particles or fines at the bottom of the container. The air escapes out the top of the chamber drawn by the suction generated by the second ID fan 53. The fines are reintroduced to the conveyor belt at the yield recovery agglomerator.

Agglomeration drums or agglomerators are commonly used in the mineral processing industry and are also known as balling drums, rotary granulators, or drum agglomerators. Agglomeration drums work by tumbling material in a rotating drum in the presence of a binding agent. The binding agent causes the fines to become tacky and allows them to pick up additional fines, forming agglomerates in a process referred to as coalescence. The tumbling action helps to round the agglomerates and create a homogenous mixture. The use of the agglomerator, is preferred over other acceptable methods of finishing the aggregate by such as “rock on rock” impact rock boxes in producing optimum particle shapes. The preference based upon choosing methods adapted to keeping the fine modulus (“FM”) lower than the target range of 2.5 to 3.0. In the presently preferred embodiment, the keeping of the final FM at values below 3.0 enhances the final flow-ability of the finished ceramic.

Returning to the discussion of the metered gate 35, an air lance 23 stirs the contents of the bulk yardage receptacle 29 to assure an even layer of premeasured charges of blended material to a perforated chevron conveyor belt 37.

The presence of iron oxide in a concrete mixture has important influence on the strength of the resulting concrete when set. Iron oxides have a beneficial effect upon concrete in limited quantities, however, once the concentration exceeds 2.5%, the effect is deleterious. The partial replacement of the cement by iron powder improved significantly the compressive strength in comparison of the concrete without iron powder (Fe₂O₃) particles. If cement is replaced by iron oxide powder up to a maximum limit of 2.5% with predominant particles of 200 nm the resulting strength of the concrete is enhanced. Beyond 2.5%, however, the resulting concrete loses compressive strength. Also, while the porosity of the concrete decreased where oxide is introduces at proportions from 1.5% and 2.5% in the replacement of cement, and then when mixed at greater concentrations, the porosity increased. Also, the replacement of cement by iron powder improved the compressive strength and porosity to a point at a concentration of 2.5% but decreased the slump flow.

Iron oxides are among the compounds having ferromagnetic properties capable of being magnetic when placed in an external magnetic field. Iron oxide has a unique superparamagnetic property susceptible to easy separation by magnetic means. The magnetic separation times and magnetic field strength have important influence on the iron removal effect of silica sand. With the increase of magnetic separation times, the iron content decreases gradually; while most iron can be removed under certain magnetic field strength, the grade of 40 mesh SiO₂ can reach 99.05%, Fe₂O₃ content is 0.071%: the SiO₂ grade of 40-80 mesh is 99.09%, Fe₂O₃ content is 0.070%; SiO₂ grade of 80-140 mesh is 99.14%, Fe₂O₃ content is 0.070%; SiO₂ grade of 80-140 mesh is 99.14%, Fe₂O₃ content is 0 067%: the SiO₂ grade of 140-00 mesh is 99.10%, and the content of Fe₂O₃ is 0.069%. The finer the quartz sand is, the better the iron removal effect, it means that the iron impurity of silica sand is strong in inclusions. The wet magnetic separator is used to improve the magnetic contact area of silica sand and reduce the inclusion of iron impurities, so that the effect of iron removal is more obvious. Thus, at the discharge from the conveyor 37, a hydrophobic magnetic sieve 45. Electromagnetic separators use wire coils and direct current to provide a magnetic field which can be used to separate ferrous material from nonferrous products. The magnetic sieve 45 is positioned overhead suspended over the chevron belt conveyor 37 at the head pulley and is used to remove iron oxides. In a preferred embodiment, the iron oxide is attracted in a self-cleaning model and is automatically and continuously removed from the magnet face by a belt that travels around the body of the magnet.

The cement mixture, now removed of its iron oxides, falls from the chevron conveyor belt 37 into a specific gravity sensor 47. Only particulate with the designated specific gravity is admitted to a double ribbon blender 49 giving the material a final blend before the material is sent to a fluidized bed dryer 51. Such a fluidized bed dryer 51 is used for drying and cooling of now thoroughly blended concrete mixture powder. The use of the fluidized bed dryer 51 enables adjustment of process parameters in order to achieve a superior overall drying economy and powder quality. Advantageously, the fluidized bed dryer 51 is also ideal for other kinds of powder treatment such as mixing, agglomeration, dust binding and instantizing.

Finally, dried material is discharged to sealed storage 60.

While the present invention is illustrated by description of several embodiments and while the illustrative embodiments are described in detail, it is not the intention of the applicants to restrict or in any way limit the scope of the appended claims to such detail. Additional advantages and modifications within the scope of the appended claims will readily appear to those sufficed in the art. The invention in its broader aspects is therefore not limited to the specific details, representative apparatus and methods, and illustrative examples shown and described. Accordingly, departures may be made from such details without departing from the spirit or scope of the invention.

1. A method for amending out of specification ash to a suitable amended addition material to a concrete mixture, the method comprising:
analyzing a unit quantity of out of specification ash to determine concentrations of each of:
calcium oxide;
sulfur trioxide; and
unburnt carbon;
sieving the out of specification ash to determine fineness;
based upon measured concentrations and determined fineness, designing a mixture comprising added materials in design-determined quantities of each of:
the out of specification ash;
quicklime;
kaolin;
masonry silica blend;
manufactured sand; and
in specification fly ash;
adding each of the design-determined quantities of the out of specification ash; quicklime, kaolin, masonry silica blend, manufactured sand and in specification fly ash in each of the design-determined quantities to create an amalgamated blend of added materials to form an amended addition material; and substituting the amended addition material for a selected quantity of Portland cement in the concrete mixture.

2. The method of claim 1, further comprising:
blending design-determined quantities of each of the out of specification ash, quicklime, kaolin, masonry silica blend, manufactured sand, and in specification fly ash by means of a pugmill.
